SEQUENCE LISTING (4 sequences)

SEQ ID NO: 1 - protein, 332 aa, Daucus carota
  Vaffr-2d
MNIESSFCPILCICMIFLCLPNLSASQRCNNNDKQALLQIKTALKNPTITDSWVSDDDCCGWDLVECDETSNRIISLIIQDDEALTGQIPPQVGDLPYLQALWFRKLPNLFGKIPEEISALKDLKSLRLSSTSLSGPVPLFFPQLTKLTCLDLSFNKLLGVIPPQLSTLPNLKALHLERNELTGEIPDIFGNFAGSPDIYLSHNQLTGFVPKTFARADPIRLDFSGNRLEGDISFLFGPKKRLEMLDFSGNVLSFNFSRVQEFPPSLTYLDLNHNQISGSLSSELAKLDLQTFNVSDNNLCGKIPTGGNLQRFDRTAYLHNSCLCGAPLPEC

SEQ ID NO: 2 - protein, 104 aa, Chlorella vulgaris
  AFP-6
MAGNKPITEQISDAVGAAGQKVGETFEAAKAQAASLTGTAEQKATEAKHDANRQGGGVVDDIKGAAAEAQHRAGETAEKAKHNVQEGWTETKHKVDEARPNATR

SEQ ID NO: 3 - protein, 20 aa, Artificial Sequence
  PelB
MKYLLPTAAAGLLLLAAQPA

SEQ ID NO: 4 - protein, 6 aa, Artificial Sequence
  6xHis
HHHHHH

(19) United States
(12) Patent Application Publication
(10) Pub. No.: US 2024/0260586 A1
(43) Pub. Date: Aug. 8, 2024

(54) ANTI-FROST PROTEIN-BASED PLANT PROTECTION AGENTS

(71) Applicant: GLOBACHEM NV, Sint-Truiden (BE)

(72) Inventors: Guy VAN DAELE, Westerlo (BE); Lisbeth VOGELS, Kuringen (BE)

(73) Assignee: GLOBACHEM NV, Sint-Truiden (BE)

(21) Appl. No.: 18/570,504

(22) PCT Filed: Jun. 15, 2022
(86) PCT No.: PCT/EP2022/066416

§ 371 Date: Dec. 14, 2023

(30) Foreign Application Priority Data
Jun. 15, 2021 (EP) 21179 554.7

(51) Int. Cl. A01N 63/50; A01P 3/00; A01P 7/04

(52) U.S. Cl. CPC A01N 63/50; A01P 3/00; A01P 7/04

(57) ABSTRACT

The present invention relates to anti-frost proteins, and in particular for a use thereof in plant protection. The invention also relates to a nucleic acid encoding a anti-frost protein a method for producing a anti-frost protein, a plant comprising a anti-frost protein or a nucleic acid encoding the same, a composition of at least one anti-frost protein. The invention relates in particular to the use of an anti-frost protein or a composition comprising the same as a plant protection agent and to a method of protecting plants from pests and/or abiotic stress.

### CROSS REFERENCE TO RELATED APPLICATIONS

The present application is a U.S. National Phase Application under 35 U.S.C. § 371 of International Patent Application No. PCT/EP2022/066416 filed Jun. 15, 2022, which claims priority of European Patent Application No. 21 179 554.7 filed Jun. 15, 2021. The entire contents of which are hereby incorporated by reference.

### FIELD OF INVENTION

The present invention relates to anti-frost proteins, and in particular for a use thereof in plant protection. The invention also relates to a nucleic acid encoding an anti-frost protein, a method for producing an anti-frost protein, a plant comprising an anti-frost protein or a nucleic acid encoding the same, a composition of at least one anti-frost protein. The invention relates in particular to the use of an anti-frost protein or a composition comprising the same as a plant protection agent and to a method of protecting plants.

### BACKGROUND

Abiotic stress, such as frost or drought, is a major concern in the agricultural industry, as it is not always possible for a plant to adjust appropriately to coldness, drought, salt salinity, heat, toxins, etc. Abiotic stress describes the negative impact of non-living factors on living organisms. Abiotic stress factors are naturally occurring factors such as intense sunlight, temperature or wind that may cause harm to the organisms. Abiotic stress affects animals as well, but plants are especially dependent on environmental factors and cannot actively change location, and are thus particularly prone to abiotic stress. Abiotic stress is the most harmful factor concerning the growth and productivity of crops worldwide.

For example, drought stress is one of the main causes of crop losses within the agricultural world. Likewise, frost also significantly contributes to crop loss, at least in those regions that do not have temperatures above freezing temperature all year.

One important way that plants combat drought stress is by closing their stomata. A key hormone regulating stomatal opening and closing is abscisic acid. Another important factor in dealing with drought stress and regulating the uptake and export of water is aquaporins. Aquaporins are integral membrane proteins that constitute membrane channels that transport water and other necessary solutes.

One mechanism that contributes to frost damage is the formation of water crystals that can disrupt the structure and function of plant cells. Specifically, freezing causes the plant's cells to shrink, forcing water into spaces between the cells where it can freeze and form ice crystals. Membranes become disrupted and leak. As temperatures rise and thawing begins the water is absorbed back into the cells by osmosis. If this occurs quickly there is no damage to the tissue, but if thawing is slow, the cells are deprived of water and become dehydrated resulting in ‘frost burn’. Frost stress can be combated by e.g. overhead sprinkling if enough water is available, or overhead protection of the plants to retain radiant heat near the plants. Plants themselves also secrete anti-frost proteins (AFPs) to provide freezing tolerance.

The present application surprisingly shows that some AFPs are at the same time chitinases.

Chitin is a polymer of N-acetylglucosamine, a derivative of the saccharide glucose, with the chemical formula (C₈H₁₃O₅N)ₙ. The long-chain polysaccharide occurs in a wide variety of different organisms across different clades. For example, chitin is a primary component of cell walls of fungi, the exoskeletons of arthropods, such as crustaceans and insects, the radulae of mollusks, and the scales of fish. Chitin has a structure that is comparable to cellulose.

The biological conversion of chitin polysaccharides into shorter oligomers requires hydrolytic enzymes that contain conserved chitin-binding domains and chitin-specific active sites. Many chitinolytic enzymes are produced by a variety of bacteria and fungi for degradation of chitin as energy source. All of them are glycosyl hydrolases, but they differ in terms of reaction mechanism, thermostability and product characteristics [Patil et al., Enzyme Microb. Technol., 2000. 26: p. 473-483]. Chitinolytic hydrolases can be categorized according to their mode of action. Endo-chitinases (EC 3.2.1.14) bind randomly to a chitin polysaccharide strand and hydrolyze internal glycosidic bonds producing various fragment sizes ranging from dimers to polymers. In contrast, exo-chitinases (EC 3.2.1.29) bind to the reducing or non-reducing end of chitin and release monomeric and to lesser extent dimeric GlcNAc units. These enzymes are necessary for the complete degradation of chitin. Finally, chitobiases (EC 3.2.1.29) cleave GlcNAc dimers to release GlcNAc monomers [Tews et al., Nat. Struct. Biol., 1996. 3: p. 638-648]. Other enzymes such as cellulase and lysozyme are also known to exhibit some hydrolytic activity towards chitin but are not specific for these substrates [Wu et al., J Food Sci Technol, 2012. 49(6): p. 695-703; Aiba, Carbohydr Res, 1994. 261: p. 297-306].

Common pests of plants include fungi, insects and mollusks. Pest infestation can result in crop loss and contamination of agricultural products with undesired side products.

Current commercially applied chemical pesticides comprise substances of the groups of organochlorines, organophosphates, carbamates, pyrethroids, triazines and neonicotinoids for use as insecticides, herbicides, fungicides and rodenticides. These pesticides are used not only for agricultural areas, but also for non-agricultural public urban green areas, sports fields, pet shampoos, building materials or boat bottoms to eliminate or prevent the presence of unwanted species. These substances have been critically reviewed as numerous negative health effects have been associated with chemical pesticides and high occupational, intentional or accidental exposure can result in hospitalization or death, whereas exposure occurs via skin contact, ingestion of contaminated consumables or inhalation upon which they may be metabolized, excreted, stored or accumulated in the body fat. [Nicolopoulou-Stamati et al., Front. Public Health, 2016, 4:148]

An ideal pesticide should not only be non-hazardous to the human health, but should also be environmentally friendly and as efficient and specific as possible for protecting a plant from a given pest. Moreover, a pesticide should ideally avoid development of a resistance in pests. There is still a need for new products that fulfil these criteria.

The present application thus deals both with the protection from abiotic stress and biotic stress (such as from pests) using AFPs.

### DESCRIPTION OF INVENTION

The present invention aims to overcome the issues of current plant protection agents by providing an anti-frost protein-based approach against abiotic stress, and in particular frost stress and drought stress, and/or on pest control. Specifically, the inventive approach relies on anti-frost proteins for protecting plants from biotic and/or abiotic stress by inducing the activation of defense mechanisms in the plant (i.e., by inducing plant immunity). Plants have an immune system that allows defending themselves against a wide range of pathogens and abiotic stresses. Some anti-frost proteins may also specifically degrade chitin, which is not produced in humans or other higher animals, and is thus expected to not pose a risk for human consumption or for other non-target organisms. Moreover, the anti-frost proteins are fully biologically degradable and thus environmentally friendly. Apart from this, given that chitin is a central structural component in pests such as fungi or insects, as well as in the radula of mollusks, such pests are not expected to easily develop a resistance against anti-frost proteins with chitinolytic activity.

The present inventors have found out that some anti-frost proteins activate the plant immunity system. The plants become by this mechanism more resistant to abiotic stress or to certain pests such as fungi or insects.

The present inventors have also found out that some anti-frost proteins having chitinolytic activity can indeed be used for protecting plants from pests such as fungi, insects and abiotic stress.

The present application shows for the first time how an anti-frost protein is applied as plant protection agent to counter pest infestation, e.g. via application of the anti-frost proteins to a surface of a plant or a part thereof.

Further, the invention makes an important contribution to the prior art plant protection agents, as the anti-frost proteins and their ensuing use as plant protection agent offers distinct advantages when compared to established substances. Such advantages may include the absence of safety hazards during handling or absence of pathogenicity upon entry into the food chain. Further, when compared to established substances, much lower concentrations of the anti-frost proteins in accordance with the present invention are needed to achieve plant protection. This may lead to cost savings.

Finally, the present inventors have found that application of the anti-frost proteins in accordance with the present invention can be combined with the application of prior art plant protection agents, thereby further enhancing plant protection.

Accordingly, the present invention provides the following preferred embodiments:
[1] A composition comprising at least one anti-frost protein, the anti-frost protein comprising a first amino acid sequence that is at least 70%, such as 100%, identical to an amino acid sequence selected from the group of SEQ ID NOs: 1-2.
[2] The composition according to [1], wherein the first amino acid sequence is at least 70%, such as 100%, identical to the amino acid sequence according to SEQ ID NO: 1.
[3] The composition according to [1] or [2], wherein the anti-frost protein comprises the amino acid sequence according to SEQ ID NO: 1.
[4] The composition according to [1] wherein the first amino acid sequence is at least 70%, such as 100%, identical to the amino acid sequence according to SEQ ID NO: 2.
[5] The composition according to [1] or [4], wherein the anti-frost protein comprises the amino acid sequence according to SEQ ID NO: 2.
[6] The composition according to any one of [1]-[5], wherein the anti-frost protein essentially consists of a first amino acid sequence that is at least 70%, such as 100%, identical to an amino acid sequence selected from the group of SEQ ID NOs: 1-2.
[7] The composition according to any one of [1]-[6], wherein the anti-frost protein consists of a first amino acid sequence that is at least 70%, such as 100%, identical to an amino acid sequence selected from the group of SEQ ID NOs: 1-2.
[8] The composition according to any one of [1]-[7] that is a plant protection agent.
[9] The composition according to any one of [1]-[8], wherein the composition is a liquid composition.
[10] The composition according to any one of [1]-[9], wherein the composition is an aqueous composition.
[11] The composition according to any one of [1]-[10], wherein the anti-frost protein is comprised in the composition at a concentration of from 0.01 mg/l (w/v) to 100 mg/l (w/v).
[12] The composition according to any one of [1]-[11], wherein the anti-frost protein is comprised in the composition at a concentration of from 0.1 mg/l (w/v) to 70 mg/l (w/v).
[13] The composition according to any one of [1]-[12], wherein the anti-frost protein is comprised in the composition at a concentration of from 5 mg/l (w/v) to 30 mg/l (w/v).
[14] The composition according to any one of [1]-[13], wherein the composition comprises polyvinyl alcohol.
[15] The composition according to any one of [1]-[14], wherein the composition comprises polyvinyl alcohol at a concentration of from 6% (v/v) to 10% (v/v), preferably at a concentration of about 8% (v/v).
[16] Use of a composition comprising at least one anti-frost protein as a plant protection agent.
[17] Use of the composition according to [16], wherein the composition is according to any one of [1]-[15].
[18] Use of the composition according to or as a plant protection agent against abiotic stress and/or a pest.
[19] Use of the composition according to any one of [16]-[18], wherein the plant protection agent is against abiotic stress, wherein the abiotic stress optionally is frost stress or drought stress.
[20] Use of the composition according to any one of [16]-[19], wherein the plant protection agent is against abiotic stress, wherein the abiotic stress is frost stress.
[21] Use of the composition according to any one of [16]-[20], wherein the plant protection agent is against abiotic stress, wherein the abiotic stress is drought stress.
[22] Use of the composition according to any one of [16]-[18], wherein the plant protection agent is against a pest, wherein the pest optionally is an organism that contains chitins, such as a fungus or an insect.
[23] Use of the composition according to any one of [16]-[18] and [22], wherein the plant protection agent is against a pest, wherein the pest optionally is a fungus or an insect.
[24] Use according to any one of [16]-[18] and [22]-[23], wherein the pest is a fungus.
[25] Use according to any one of [22]-[24], wherein the fungus is a Puccinia, Fusiarum or Septoria species.
[26] Use according to any one of [22]-[25], wherein the fungus is a Fusiarum or Septoria species.
[27] Use according to any one of [22]-[26], wherein the fungus is a Fusiarum species, preferably Fusarium culmorum.
[28] Use according to any one of [22]-[24] and [25], wherein the fungus is a Puccinia species.
[29] Use according to any one of [22]-[25] and [28], wherein the fungus is Puccinia triticina.
[30] Use according to any one of [16]-[29], wherein the plant is an arable crop, fruit-bearing plant or vegetable.
[31] Use according to any one of [16]-[30], wherein the plant is an arable crop such as wheat.
[32] Use according to any one of [16]-[31], wherein the composition is applied on a plant or a part thereof.
[33] Use according to any one of [16]-[32], wherein the composition is applied on a plant or a part thereof before the abiotic stress and/or the pest occurs.
[34] Use according to any one of [16]-[33], wherein the part of a plant is a leaf, a fruit or a seed.
[35] Use according to any one of [16]-[34], wherein the part of a plant is a leaf.
[36] Use according to any one of [16]-[34], wherein the part of a plant is a seed.
[37] Use according to [36], wherein the composition is applied on the seed before sowing.
[38] Use according to any one of [16]-[37], wherein the composition induces plant immunity.
[39] Use according to any one of [16]-[38], wherein one or more further plant protection agent(s) is/are applied on the plant or a part thereof, wherein the further plant protection agent(s) is/are not an anti-frost protein.
[40] A method of protecting a plant from abiotic stress and/or a pest, the method comprising the application of a composition comprising at least one anti-frost protein on the plant or a part thereof.
[41] The method of protecting a plant according to [40], wherein the composition is according to any one of [1]-[15].
[42] The method of protecting a plant according to or [41], wherein the composition is applied on the plant or a part thereof before the abiotic stress and/or the pest occurs.
[43] The method of protecting a plant according to any one of [40]-[42], wherein the part of a plant is a leaf, a fruit or a seed.
[44] The method of protecting a plant according to any one of [40]-[43], wherein the part of a plant is a leaf.
[45] The method of protecting a plant according to any one of [40]-[43], wherein the part of a plant is a seed.
[46] The method of protecting a plant according to [45], wherein the composition is applied on the seed before sowing.
[47] The method of protecting a plant according to any one of [40]-[46], wherein the plant or plant part is soaked in the composition.
[48] The method of protecting a plant according to any one of [40]-[47], wherein the composition is applied by spraying to a surface of the plant or part of the plant, such as a seed, for example a coated a seed.
[49] The method according to any one of [40]-[48], wherein the method is for protecting the plant from abiotic stress, wherein the abiotic stress optionally is frost stress or drought stress.
[50] The method according to any one of [40]-[49], wherein the method is for protecting the plant from abiotic stress, wherein the abiotic stress optionally is frost stress.
[51] The method according to any one of [40]-[50], wherein the method is for protecting the plant from abiotic stress, wherein the abiotic stress optionally is drought stress.
[52] The method according to any one of [40]-[48], wherein the method is for protecting the plant from a pest, wherein the pest optionally is a fungus or an insect.
[53] The method according to any one of [40]-[48] and [52], wherein the pest is an organism that contains chitin, such as a fungus or an insect.
[54] The method according to any one of [40]-[48] and [52]-[53], wherein the pest is a fungus.
[55] The method according to any one of [52]-[54], wherein the fungus is a Puccinia, Fusiarum or Septoria species.
[56] The method according to any one of [52]-[55], wherein the fungus is a Fusiarum or Septoria species.
[57] The method according to any one of [52]-[56], wherein the fungus is a Fusiarum species, preferably Fusarium culmorum.
[58] The method according to any one of [52]-[55], wherein the fungus is a Puccinia species.
[59] The method according to any one of [52]-[55] and [58], wherein the fungus is Puccinia triticina.
[60] The method according to any one of [40]-[59], wherein the plant is an arable crop, fruit-bearing plant or vegetable.
[61] The method according to any one of [40]-[60], wherein the plant is an arable crop such as wheat.
[62] The method according to any one of [40]-[61], wherein the composition induces plant immunity.
[63] The method according to any one of [40]-[62], the method further comprising the application of one or more further plant protection agent(s) on the plant or a part thereof, wherein the further plant protection agent(s) is/are not an anti-frost protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. Use of AFPs with chitinases activity as plant protection agent. F. culmorum growth and plant health was assessed in the presence of an AFP with chitinase activity. FIG. 1A Control; FIG. 1B Control+F. culmorum; FIG. 1C Vaffr-2d+F. culmorum.

FIG. 2. Protective activity (damage decrease) of anti-frost proteins against frost stress. Tested by foliar application of the anti-frost protein Vaffr-2d. Results are normalized against control (CTL)

FIG. 3. Use of Vaffr-2d as plant protection agent against abiotic stresses. Yield of pears after foliar application in presence of frost stress.

FIG. 4. AFP-6 as inducer of plant immunity. % efficacy as seed treatment against Puccinia triticina in wheat, Benchmark.

FIGS. 5A-5B. AFP-6 as inducer of plant immunity. FIG. 5A % efficacy of AFP-6 as seed treatment against Puccinia triticina in wheat, Keitum. FIG. 5B % efficacy of Difend extra as seed treatment against Puccinia triticina in wheat, Keitum.

FIGS. 6A-6D. Effects of AFP-6 on jasmonic acid and salicylic acid pathways. Seeds were treated with AFP-6 at 0.1 g/ton or 0.175 g/ton, and samples of the seedlings were taken 4, 7, 9 and 14 days post seeding (dps). FIG. 6A Fold-changes in LOX mRNA levels are shown, normalized to actin (top panel) or ubiquitin (bottom panel). FIG. 6B Fold-changes in OPR3 mRNA levels are shown, normalized to actin (top panel) or ubiquitin (bottom panel). FIG. 6C Fold-changes in PR1-3 mRNA levels are shown, normalized to actin (top panel) or ubiquitin (bottom panel). FIG. 6D Fold-changes in PR1-17 mRNA levels are shown, normalized to actin (top panel) or ubiquitin (bottom panel).

FIGS. 7A-7D. Effects of Vaffr-2d on jasmonic acid and salicylic acid pathways. Seeds were treated with Vaffr-2d at 0.175 g/ton, and samples of the plants were taken 6, 7, 8 and 11 days after sowing. FIG. 7A Fold-changes in OPR3 mRNA levels are shown, normalized to actin (top panel) or ubiquitin (bottom panel). FIG. 7B Fold-changes in LOX mRNA levels are shown, normalized to actin (top panel) or ubiquitin (bottom panel).

FIG. 7C Fold-changes in PR1-3 mRNA levels are shown, normalized to actin (top panel) or ubiquitin (bottom panel). FIG. 7D Fold-changes in PR1-17 mRNA levels are shown, normalized to actin (top panel) or ubiquitin (bottom panel).

### DETAILED DESCRIPTION OF INVENTION

Unless specifically defined herein, all technical and scientific terms used herein have the same meaning as commonly understood by a skilled artisan in the fields of enzymology, plant protection, biochemistry, genetics, and molecular biology.

All methods and materials similar or equivalent to those described herein can be used in the practice or testing of the present invention, with suitable methods and materials being described herein.

The term “about” when used in the context of the present invention means that the value following the term “about” may vary within the range of +/−20%, preferably in the range of +/−15%, more preferably in the range of +/−10%.

All publications, patents and patent applications cited herein are hereby incorporated by reference in their entirety for all purposes. In case of conflict, the present specification, including definitions, will prevail over the cited references. Further, the materials, methods, and examples are illustrative only and are not intended to be limiting, unless otherwise specified.

As used herein, each occurrence of terms such as “comprising” or “comprises” may optionally be substituted with “consisting of” or “consists of”. The term “essentially consists of” in the context of compounds or compositions means that specific further components can be present that do not materially affect the essential characteristics of the compound or composition. For example, an anti-frost protein essentially consisting of a certain amino acid sequence can consist of said amino acid sequence and additional N- and/or C-terminal sequences that do not materially affect the chitinolytic activity of the enzyme.

The present invention aims to overcome the issues of current plant protection agents by providing a protein-based approach against abiotic stress and/or on pest control. Specifically, the inventive approach relies on anti-frost proteins for protecting plants. These proteins induce activation of plant defense pathways against stresses and some may also specifically degrade chitin, which is not produced in humans or other higher animals and are thus expected to not pose a risk for human consumption or for other non-target organisms. In the present application, the term “chitinolytic enzyme” is used synonymously with the term “chitinase”.

The present disclosure is described in more detail as follows.

### Anti-Frost Proteins

The present invention relates to an anti-frost protein, a composition comprising one or more anti-frost proteins, as well as to a plant protection agent, comprising or (essentially) consisting of one or more anti-frost proteins. The invention particularly relates to AFPs of SEQ ID NO: 1 or 2, variants thereof or polypeptides comprising the same. SEQ ID NO: 1 defines an AFP derived from carrots, also termed Vaffr-2d. Surprisingly, the inventors have found that Vaffr-2d also has chitinolytic activity. SEQ ID NO: 2 defines an AFP termed AFP-6.

An anti-frost protein as disclosed herein can comprise a first amino acid sequence that is at least 70% identical (such as 100% identical) to an amino acid sequence selected from the group of SEQ ID NOs: 1-2. For example, the first amino acid sequence is at least 80%, at least 90%, at least 95%, at least 98% or at least 99% identical to an amino acid sequence selected from the group of SEQ ID NOs: 1-2. Preferably, the first amino acid sequence is at least 98%, or at least 99% identical, and most preferably 100% identical to an amino acid sequence selected from the group of SEQ ID NOs: 1-2. Thus, the protein can comprise a first amino acid sequence selected from the group of SEQ ID NOs: 1-2.

In line with this, a preferred anti-frost protein can also essentially consist of a first amino acid sequence that is at least 70% identical (such as 100% identical) to an amino acid sequence selected from the group of SEQ ID NOs: 1-2. For example, the first amino acid sequence is at least 80%, at least 90%, at least 95%, at least 98% or at least 99% identical to an amino acid sequence selected from the group of SEQ ID NOs: 1-2. Preferably, the first amino acid sequence is at least 98%, or at least 99% identical, and most preferably 100% identical to an amino acid sequence selected from the group of SEQ ID NOs: 1-2. Thus, the protein can essentially consist of a first amino acid sequence selected from the group of SEQ ID NOs: 1-2.

Accordingly, an anti-frost protein can also consist of a first amino acid sequence that is at least 70% identical (such as 100% identical) to an amino acid sequence selected from the group of SEQ ID NOs: 1-2. For example, the first amino acid sequence is at least 80%, at least 90%, at least 95%, at least 98% or at least 99% identical to an amino acid sequence selected from the group of SEQ ID NOs: 1-2. Preferably, the first amino acid sequence is at least 98%, or at least 99% identical, and most preferably 100% identical, to an amino acid sequence selected from the group of SEQ ID NOs: 1-2. Thus, the protein can consist of a first amino acid sequence selected from the group of SEQ ID NOs: 1-2.

For example, the first amino acid sequence can be at least 70% identical to SEQ ID NO: 1.

For example, the first amino acid sequence can be at least 70% identical to SEQ ID NO: 2.

An anti-frost protein as disclosed herein can comprise a first amino acid sequence that exhibits up to 15 amino acid differences (such as no amino acid differences) to an amino acid sequence selected from the group of SEQ ID NOs: 1-2. For example, the first amino acid sequence exhibits up to 10, up to 5, or up to 3, 2 or 1 amino acid differences to an amino acid sequence selected from the group of SEQ ID NOs: 1-2. Preferably, the first amino acid sequence exhibits up to 3, 2 or 1 amino acid differences, most preferably no amino acid differences, to an amino acid sequence selected from the group of SEQ ID NOs: 1-2. Thus, the protein can comprise a first amino acid sequence selected from the group of SEQ ID NOs: 1-2.

In line with this, an anti-frost protein as disclosed herein can essentially consist of a first amino acid sequence that exhibits up to 15 amino acid differences (such as no amino acid differences) to an amino acid sequence selected from the group of SEQ ID NOs: 1-2. For example, the first amino acid sequence exhibits up to 10, up to 5, or up to 3, 2 or 1 amino acid differences to an amino acid sequence selected from the group of SEQ ID NOs: 1-2. Preferably, the first amino acid sequence exhibits up to 3, 2 or 1 amino acid differences, most preferably no amino acid differences, to an amino acid sequence selected from the group of SEQ ID NOs: 1-2. Thus, the protein can essentially consist of a first amino acid sequence selected from the group of SEQ ID NOS: 1-2.

Accordingly, an anti-frost protein as disclosed herein can consist of a first amino acid sequence that exhibits up to 15 amino acid differences (such as no amino acid differences) to an amino acid sequence selected from the group of SEQ ID NOs: 1-2. For example, the first amino acid sequence exhibits up to 10, up to 5, or up to 3, 2 or 1 amino acid differences to an amino acid sequence selected from the group of SEQ ID NOs: 1-2. Preferably, the first amino acid sequence exhibits up to 3, 2 or 1 amino acid differences, most preferably no amino acid differences, to an amino acid sequence selected from the group of SEQ ID NOs: 1-2. Thus, the protein can consist of a first amino acid sequence selected from the group of SEQ ID NOs: 1-2.

For example, the first amino acid sequence can exhibit up to 15 amino acid differences to SEQ ID NO: 1.

For example, the first amino acid sequence can exhibit up to 15 amino acid differences to SEQ ID NO: 2.

When the first amino acid sequence is less than 100% identical and/or has amino acid differences to a reference amino acid sequence as defined above, the anti-frost protein preferably has the same or a better protective effect (e.g. against frost and/or drought stress) as a corresponding anti-frost protein (essentially) consisting of the reference sequence. For example, when the first amino acid is at least 70% (and less than 100%) identical to the amino acid sequence of SEQ ID NO: 1, the anti-frost protein preferably has the same or a better protective effect as an anti-frost protein (essentially) consisting of SEQ ID NO: 1. Likewise, for example, when the first amino acid has up to 15 (and at least 1) amino acid differences to the amino acid sequence of SEQ ID NO: 1, the anti-frost protein preferably has the same or a better protective effect as an anti-frost protein (essentially) consisting of SEQ ID NO: 1. The same applies mutatis mutandis to SEQ ID NO: 2.

When the first amino acid sequence is less than 100% identical and/or has amino acid differences to a reference amino acid sequence as defined above, the anti-frost protein with chitinolytic activity preferably has the same or a better degradation rate as a corresponding anti-frost protein with chitinolytic activity (essentially) consisting of the reference sequence. For example, when the first amino acid is at least 70% (and less than 100%) identical to the amino acid sequence of SEQ ID NO: 1, the anti-frost protein with chitinolytic activity preferably has the same or a better chitin degradation rate as an anti-frost protein with chitinolytic activity (essentially) consisting of SEQ ID NO: 1. Likewise, for example, when the first amino acid has up to 15 (and at least 1) amino acid differences to the amino acid sequence of SEQ ID NO: 1, the anti-frost protein with chitinolytic activity preferably has the same or a better chitin degradation rate as an anti-frost protein with chitinolytic activity (essentially) consisting of SEQ ID NO: 1.

When the first amino acid sequence is less than 100% identical and/or has amino acid differences to an amino acid sequence of SEQ ID NO: 1, the skilled person knows how to modify the original sequence in order to maintain or improve the protective effect (e.g. against frost and/or drought stress) and/or the chitin degradation rate compared to the reference, i.e. unmodified, sequence (essentially) consisting of an amino acid sequence. Protective effects can be determined e.g. by spraying parts of a plant with an aqueous solution of the AFP in question (or the same solution without the AFP as control) and incubating the plant at freezing temperatures (for frost stress) or conditions with limiting water supply (for drought stress). The chitin degradation rate can be determined e.g. by incubating chitin powder as substrate with an aqueous solution of the AFP in question. Typically, the same method is used to determine the effects of a modified enzyme and that of the reference sequence.

The percentage of “sequence identity” or “% identical” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (i.e. at a single position). The same applies mutatis mutandis to nucleotide sequences.

An “amino acid difference” as used herein can be an amino acid insertion, deletion or substitution, and is preferably a substitution. An amino acid substitution is preferably a conservative substitution as known in the art. Such a conservative substitution can be a substitution in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, IIe, Val and Cys; and (e) aromatic residues: Phe, Tyr and Trp.

More specifically, a conservative substitutions can be as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; IIe into Leu or into Val; Leu into IIe or into Val; Lys into Arg, into Gln or into Glu; Met into Leu, into Tyr or into IIe; Phe into Met, into Leu or into Tyr; Ser into Thr; Thr into Ser; Trp into Tyr; Tyr into Trp; and/or Phe into Val, into IIe or into Leu.

An anti-frost protein with chitinolytic activity can be an endo-chitinase or an exo-chitinase. Preferably, the anti-frost protein with chitinolytic activity is capable of cleaving chitin that is present as a structural component of a fungus and/or an insect. A structural component of a fungus can be the cell wall. A structural component of an insect can be the exoskeleton. Most preferably, the anti-frost protein with chitinolytic activity is capable of cleaving chitin that is present in the cell wall of a fungus.

The anti-frost protein can further comprise a second amino acid sequence fused to the N-terminus of the first amino acid sequence. The second amino acid sequence is typically located at the N-terminus of the protein.

The second amino acid sequence preferably is less than 50 amino acids in length, more preferably less than 30, even more preferably less than 25 amino acids, such as 22 amino acids.

The second amino acid sequence is typically a sequence that causes secretion from a cell, such as a bacterial cell. Accordingly, the second amino acid can be a signal peptide. Specific examples of the second amino acid sequence include a PelB signal peptide (SEQ ID NO: 3).

The anti-frost protein can further comprise a third amino acid sequence fused to C-terminus to the first amino acid sequence. The third amino acid sequence is typically located at the C-terminus of the protein.

The third amino acid sequence is preferably less than 50 amino acids in length, more preferably less than 30, even more preferably less than 20 amino acids. Most preferably, the third amino acid sequence is less than 10 amino acids in length, such as 6 amino acids.

The third amino acid sequence is typically a sequence that facilitates purification of the anti-frost protein after production by a cell, such as a bacterial cell. Accordingly, the third amino acid can be a purification tag. Specific examples of a purification tag include a 6×His tag (SEQ ID NOs: 4) or a Tag54/6×His combi-tag. Preferably, the third amino acid sequence is a 6×His tag.

The invention thus also provides an anti-frost protein comprising or (essentially) consisting of a first amino acid sequence, a second amino acid sequence and a third amino acid sequence, wherein the second amino acid sequence is a PelB signal peptide and the third amino acid sequence is a 6×His tag.

The anti-frost protein is preferably a purified anti-frost protein. “Purified” in this context means that less than 5% of impurities are present, such as less than 2% or even less than 1% impurities. Impurities in this context means any substances other than the protein and optionally a solvent.

Particularly preferred is an anti-frost protein comprising SEQ ID NO: 1 or an amino acid sequence having at least 70% sequence identity or up to 15 amino acid differences thereto.

Particularly preferred is also an anti-frost protein comprising SEQ ID NO: 2 or an amino acid sequence having at least 70% sequence identity or up to 15 amino acid differences thereto.

### Nucleic Acid, Vector and Host (Cell)

The invention also relates to a nucleic acid encoding an anti-frost protein. More specifically, the invention provides a nucleic acid encoding an anti-frost protein as described herein.

A nucleic acid encoding an anti-frost protein can also encode more than one protein as described herein. Thus, the invention provides a nucleic acid encoding an AFP comprising a first amino acid sequence that is at least 70% identical (such as 100% identical) to SEQ ID NO: 1 and an AFP comprising a first amino acid sequence that is at least 70% identical (such as 100% identical) to SEQ ID NO: 2.

A nucleic acid may be for example DNA, RNA, or a hybrid thereof, and may also comprise (e.g. chemically) modified nucleotides, like PNA. It can be single- or double-stranded DNA. For example, the nucleotide sequences of the present disclosure may be genomic DNA, cDNA.

The invention further provides a vector comprising the nucleic acid encoding an anti-frost protein. A vector as used herein is a vehicle suitable for carrying genetic material into a cell. A vector includes naked nucleic acids, such as plasmids or mRNAs, or nucleic acids embedded into a bigger structure, such as liposomes or viral vectors.

Vectors generally comprise at least one nucleic acid that is optionally linked to one or more regulatory elements, such as for example one or more suitable promoter(s), enhancer(s), terminator(s), etc.). The vector can be an expression vector, i.e. a vector suitable for expressing an encoded polypeptide or construct under suitable conditions, e.g. when the vector is introduced into a (e.g. bacterial or plant) cell. For DNA-based vectors, this usually includes the presence of elements for transcription (e.g. a promoter and a polyA signal) and translation (e.g. Kozak sequence).

In the vector, said at least one nucleic acid and said regulatory elements can be “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor).

Also, preferably the nucleic acid encoding an anti-frost protein may constitute part of an expression system, wherein the nucleic acid represents an open reading frame. The open reading frame may be codon-optimized for a particular organism.

The invention further provides a (non-human) host or a host cell that comprises the nucleic acid or the vector. A suitable host cell can be a plant cell or microbial cell.

For example, a plant cell from an agricultural or ornamental plant can be used. A microbial cell can be, for example, a yeast or bacterial cell, such as E. Coli. An example of a suitable yeast is Pichia pastoris.

Also provided is a plant comprising an anti-frost protein as described herein, a nucleic acid encoding the same, or a vector comprising the nucleic acid. Preferably, the nucleic acid or vector may be comprised in the genome of the plant. Examples of plants include arable crops such as wheat, fruit-bearing plants or vegetables. Examples of plants include cereals, maize, oil seed rape, rice, soy bean or potato.

Typically, the plant (cell) will be such that it does not naturally contain a gene encoding SEQ ID NO: 1 or SEQ ID NO: 2. For example, the plant (cell) is not Daucus carota. For example, the plant (cell) is not a green algae, such as Chlorella vulgaris.

### Production Method

The invention also provides a method for producing an anti-frost protein as described herein. Typically, the method comprises at least the step of culturing a host cell as described herein, and in particular a bacterial host cell, such as E. coli. The culture can be conducted in a medium suitable for growth of the host cell.

The method can further comprise a step of harvesting the host cell and/or the culture supernatant during and/or after the culture. Preferably, the supernatant is harvested after (a suitable period of) the culture.

The method can further comprise a step of purifying the anti-frost protein. For example, the anti-frost protein can be purified from the culture supernatant by an initial ammonium sulfate precipitation step and a sequential immobilized metal affinity chromatography purification of solubilized protein precipitate.

A method for producing an anti-frost protein can, for example, comprise setting up an expression system comprising culturing a host cell which expresses one or more anti-frost proteins.

Preferably the anti-frost protein produced by the method comprises N- and/or C-terminal modifications to facilitate secretion of the enzyme into the culture medium and/or purification from a culture supernatant. For example, the anti-frost protein produced by the method can comprise a second and/or a third amino acid sequence as described herein.

Accordingly, the method can comprise purifying the anti-frost protein from the culture supernatant by an initial ammonium sulfate precipitation step, wherein a sequential immobilized metal affinity chromatography purification of solubilized protein precipitate is performed using an amino acid tag, such as a 6×His tag, comprised in the enzyme.

### Composition

The invention also provides a composition comprising at least one of the anti-frost proteins as described herein. Preferably, the composition induces plant immunity.

Preferably the composition comprises at least two different anti-frost proteins as described herein.

The composition may for example comprise an anti-frost protein as described herein comprising SEQ ID NO: 1 and an anti-frost protein as described herein comprising SEQ ID NO: 2.

The composition may for example comprise an anti-frost protein as described herein essentially consisting of SEQ ID NO: 1 and an anti-frost protein as described herein essentially consisting of SEQ ID NO: 2.

The composition may for example comprise an anti-frost protein as described herein consisting of SEQ ID NO: 1 and an anti-frost protein as described herein consisting of SEQ ID NO: 2.

The composition can be a liquid or a dry composition, preferably a liquid composition. A liquid composition can suitably be an aqueous composition. The composition may comprise polyvinyl alcohol (PVOH), which is a standard sticker used in commercial seed treatments. The concentration of the polyvinyl alcohol in the composition may be, e.g., from 5% (v/v) to 15% (v/v), preferably from 6% (v/v) to 10% (v/v), more preferably about 8% (v/v).

Typically, the composition does not comprise an extract from plants that naturally contain a gene encoding SEQ ID NO: 1 or SEQ ID NO: 2. For example, the composition may be a composition that does not comprise an extract from Daucus carota, or a green algae, such as Chlorella vulgaris.

The concentrations of the anti-frost protein (e.g., the chitinolytic enzyme) in the composition may be e.g. 0.01 mg/L to 250 g/L, such as 0.025 mg/L to 100 g/L. Alternatively, the concentrations of the anti-frost protein (e.g., the chitinolytic enzyme) in the composition may be, e.g., up to 1 g/l (w/v), such as up to 100 mg/l (w/v), or from 0.01 mg/l (w/v) to 100 mg/l (w/v), such as from 0.1 mg/l (w/v) to 70 mg/l (w/v), or from 1 mg/l (w/v) to 50 mg/l (w/v), or from 5 mg/l (w/v) to 30 mg/l (w/v), or from 7 mg/l (w/v) to 25 mg/l (w/v), or about 9 mg/l (w/v), or about 13 mg/l (w/v), or about 22 mg/l (w/v). Further specific examples of concentrations are as follows, according to applications as further described herein:
Insecticidal application: 0.01% to 5% (w/v), such as 0.05% to 2.5% (w/v), preferably 0.1 to 1% (w/v)
Fungicidal application: 0.25 μg/100 μl to 25.0 μg/100 μl, such as 0.7 μg/100 μl to 15.0 μg/100 μl, preferably 1.25 μg/100 μl to 10.0 μg/100 μl
Indirect fungicidal application: 0.25 μg/100 μl to 25.0 μg/100 μl, such as 0.7 μg/100 μl to 15.0 μg/100 μl, preferably 0.65 μg/100 μl to 5 μg/100 μl
Abiotic stress: 0.01 mg/L to 1 mg/L, such as 0.025 mg/L to 0.5 mg/L, preferably 0.05 mg/L to 0.25 mg/L

Preferably, the composition does not comprise an inhibitor of protective effects and/or chitinolytic activity. Such inhibitors may be metallic ions (for example, divalent ions, such as Zn2+, Cu2+, Ni2+), detergents (for example, sodium dodecyl sulfate (SDS), Triton X100 or Polysorbate 20), or certain other chemicals (for example, EDTA, imidazole). Thus, for example, the composition does not comprise metallic ions and/or SDS.

### Plant Protection

The present inventors have surprisingly found that the anti-frost proteins can be used for protecting plants from different abiotic stresses, including frost and drought stress. Moreover, surprisingly, the AFPs, and in particular those having chitinolytic activity, can also be used for protecting plants from pests such as fungi.

The present inventors have also found that anti-frost proteins, and in particular AFP-6 and Vaffr-2d, activate the plant immunity system, i.e. that they induce plant immunity. The terms “induce plant immunity” and “activate the plant immunity system” are used interchangeably herein. Induction of plant immunity can be tested, for example, by testing for activation of the jasmonic acid pathway and/or the salicylic acid pathway in plants. These pathways play an important role as regulators of plant defense against biotic and abiotic stress. The skilled person is well aware of how to test for activation of the jasmonic acid pathway and/or the salicylic acid pathway. For example, activation of the jasmonic acid pathway can be tested by assessing changes in the mRNA levels of the LOX and/or OPR3 genes, which are upregulated in order to activate the jasmonic acid pathway (Chini, 2018: “An OPR3-independent pathway uses 4,5-didehydrojasmonate for jasmonate synthesis”; Nature chemical biology, 14(2), 171-178; doi: https://doi.org/10.1038/nchembio.2540) (León, 1999: “Molecular biology of jasmonic acid biosynthesis in plants”; Plant physiology and d Biochemistry, 37(5), 373-380; doi: https://doi.org/10.1016/S0981-9428(99)80043-6). Activation of the salicylic acid pathway can be tested by assessing changes in the mRNA levels of the PR1-3 and/or PR1-17 genes. Production of the PR1-3 and PR1-17 proteins is linked with the plant hormone salicylic acid (Van Loon, 1999: “The families of pathogenesis-related proteins, their activities, and comparative analysis of PR-1 type proteins”; Physiological and molecular plant pathology, 55(2), 85-97; doi: https://doi.org/10.1006/pmpp.1999.0213). Exemplary methods of testing for activation of the jasmonic acid pathway and/or the salicylic acid pathway are further described in Example 7 below. The plant immunity system is conserved in all plants, therefore the anti-frost proteins in accordance with the present invention can be assumed to activate the immunity system in all plants. Advantageously, plant immunity is induced before the plant is damaged by abiotic stress and/or a pest. Therefore, preferably the anti-frost proteins in accordance with the present invention (or a composition comprising same) are applied on the plant or a part thereof before the abiotic stress and/or the pest occurs, e.g. before the plant is damaged by the abiotic stress and/or the pest.

Further, the present inventors have found that application of the anti-frost proteins in accordance with the present invention can be combined with application of prior art plant protection agents, thereby further enhancing plant protection. Therefore, further plant protection agents which are not anti-frost proteins can be applied on the plant or a part thereof in addition to application of the anti-frost proteins in accordance with the present invention. Such further plant protection agents can be chemical pesticides such as substances of the groups of organochlorines, organophosphates, carbamates, pyrethroids, triazines and neonicotinoids, e.g. for use as insecticides, herbicides, fungicides and rodenticides. For example, such further plant protection agents can be Difend extra (25 g/l difeconazole and 25 g/l fludioxinil) and/or Protendo (prothioconazole 300 g/l EC) and/or Velogy era (EC 75 g/l benzovindiflupyr+150 g/l prothioconazole). Such further plant protection agents can also be, for example, the further agents ascorbic acid, betaine and/or salicylic acid mentioned below.

Apart from the effects on pests and abiotic stress by inducing the natural defense mechanisms in plants against fungi and abiotic stress, the chitinolytic activity of anti-frost proteins can also have direct effects on pests and diseases of plants.

The present invention thus provides a composition comprising at least one anti-frost protein as described herein that is a plant protection agent. The present invention further provides a composition comprising at least two anti-frost proteins as described herein that is a plant protection agent.

The present invention also provides a use of a composition comprising at least one anti-frost protein as described herein as a plant protection agent. The present invention also provides a use of a composition comprising at least two anti-frost proteins as described herein as a plant protection agent.

The plant protection agent is preferably for protecting the plant against abiotic stress and/or for protecting the plant against a pest, e.g. against an organism that contains chitin.

Thus, the plant protection agent can be for protecting the plant against abiotic stress. Abiotic stress include, for example, frost, drought, salt, flooding or heat stress. Preferably, the abiotic stress is frost stress or drought stress.

The plant protection agent can (also) be for protecting the plant against a pest, e.g. against organisms that contain chitin. For example, chitin is a primary component of cell walls of fungi, the exoskeletons of arthropods, such as insects and the radulae of mollusks. Accordingly, the plant protection agent can be against infestation of fungi, insects or mollusks, and preferably fungi or insects, most preferably fungi.

Examples of fungi include ascomycetes, e.g. from the family Nectriaceae or Mycosphaerellaceae. Examples of fungi from the family of Nectriaceae include fungi from the genus Fusarium, such as Fusiarum oxysporum, Fusiarum graminearum, Fusarium culmorum. Examples of fungi from the family Mycosphaerellaceae include fungi from the genus Septoria, such as Septoria tritici. Other examples include Alternaria solani, Phytophtora infestans, Pythium, Magnaporthe oryzae, Venturia inaequalis, Pyrenophora teres, Rhynchosporium secalis, Puccinia triticina and Ramularia collo-cygni. The fungi can be filamentous fungi. The fungi are typically pathogenic fungi.

Examples of insects include insects from the family Aphididae, Tenebrionidae, Drosophilidae or Aphrophoridae. Examples of insects from the family of Aphididae include insects from the genus Sitobion, such as Sitobion avanae. Examples of insects from the family of Tenebrionidae include insects from the genus Tribolium, such as Tribolium castaneum. Examples of insects from the family of Drosophilidae include insects from the genus Drosophila, such as Drosophila melanogaster. Examples of insects from the family of Aphrophoridae include insects from the genus Philaenus, such as Philaenus spumarius.

The plant to be protected is not particularly limited, and includes, for example, arable crops such as wheat, fruit-bearing plants or vegetables.

Examples of plants include cereals, maize, oil seed rape, rice, soy bean or potato.

The invention further provides a method of protecting a plant from abiotic stress and/or pests, the method comprising the application of an anti-frost protein or a composition comprising at least one anti-frost protein, such as an anti-frost protein as described herein, on the plant or a part thereof. Typically, the composition is applied to the surface of the plant or a part thereof.

Abiotic stress includes, for example, frost, drought, salt or heat stress. Preferably, the abiotic stress is frost stress or drought stress.

Pests include, for example, fungi, insects or mollusks. Examples thereof are given above. Preferably, the pest is a fungus or an insect, most preferably a fungus. The pests may be biotrophic or necrotrophic.

The application of the anti-frost protein or the composition can for example comprise soaking of a plant or a plant part in a composition as described herein. Another exemplary application of the anti-frost proteins can comprise coating of a plant or a plant part (e.g., a seed) with the anti-frost proteins, e.g. using a composition as described herein, e.g. using the coating method described in the Examples. Another exemplary application of the anti-frost proteins can comprise spraying a composition as described herein onto a plant or a plant part. For example, the anti-frost proteins may be sprayed at a dose rate of about 70 mg/ha using around 200 l water per ha (i.e., using a composition comprising anti-frost proteins at a concentration of about 0.35 mg/l), or using 1000 l or 1500 l water/ha.

A plant part can be, for example, a leaf, a fruit or a seed (such as a grain). When the plant part is a seed, the anti-frost proteins in accordance with the present invention (or a composition comprising same) are preferably applied on the seed before sowing. A seed can be a coated or an uncoated seed. Coated seed technology is commonly known and readily amendable to a person skilled in the art.

The plant to be protected is not particularly limited, and includes, for example, arable crops, fruit-bearing plants or vegetables. Examples are described above.

Plant protection may also be achieved by expressing at least one anti-frost protein as described herein in a plant or plant cell. Accordingly, the invention also provides a use of the nucleic acid or the vector as described herein for expressing an anti-frost protein in a plant or plant cells. Expression may be constitutive or inducible. For example, the expression may be inducible in response to external stimuli, e.g. abiotic stress and/or pest infestation (for example, via endogenous sensory mechanisms in the plant that can detect abiotic stress and/or tissue damage).

### Further Agents

The anti-frost proteins can be suitably combined with further agents that can act as plant protection agents, e.g. agents against abiotic stress, fungicides and/or insecticides. Such further agents can be, for example, ascorbic acid, betaine and/or salicylic acid.

Thus, the invention also provides a composition comprising at least one anti-frost protein and the uses thereof as described herein, further comprising ascorbic acid, betaine and/or salicylic acid.

The invention also provides a composition comprising at least one anti-frost protein and the uses thereof as described herein, further comprising a fungicide and/or an insecticide.

TABLE 1
Sequences

| | SEQ | |
|---|---|---|
| | ID | |
| Name | NO | Sequence |
| Vaffr- | 1 | MNIESSFCPILCICMIFLCLPNLSASQRCNNNDKQALL |
| 2d | | QIKTALKNPTITDSWVSDDDCCGWDLVECDETSNRIIS |
| | | LIIQDDEALTGQIPPQVGDLPYLQALWFRKLPNLFGKI |
| | | PEEISALKDLKSLRLSSTSLSGPVPLFFPQLTKLTCLD |
| | | LSFNKLLGVIPPQLSTLPNLKALHLERNELTGEIPDIF |
| | | GNFAGSPDIYLSHNQLTGFVPKTFARADPIRLDFSGNR |
| | | LEGDISFLFGPKKRLEMLDFSGNVLSFNFSRVQEFPPS |
| | | LTYLDLNHNQISGSLSSELAKLDLQTFNVSDNNLCGKI |
| | | PTGGNLQRFDRTAYLHNSCLCGAPLPEC |
| AFP-6 | 2 | MAGNKPITEQISDAVGAAGQKVGETFEAAKAQAASLTG |
| | | TAEQKATEAKHDANRQGGGVVDDIKGAAAEAQHRAGET |
| | | AEKAKHNVQEGWTETKHKVDEARPNATR |
| PelB | 3 | MKYLLPTAAAGLLLLAAQPA |
| 6xHis | 4 | HHHHHH |

### EXAMPLES

The following experimental section of the application relates to non-limiting exemplified embodiments of the present invention.

### Example 1

The protective activity of anti-frost proteins against fungi infestation was tested. To this end, the anti-frost proteins were produced in E. coli BL21 cells. The enzymes were purified from the culture supernatant.

The anti-frost proteins were tested for their protective activity in germination tests. Briefly, the following steps were conducted:
1. Disinfection of wheat seeds with 10% bleach for 10 minutes in a safety cabinet
2. Germination on filter paper
3. Foliar application 3 dps (days post seeding) and 1 dbi (day before infection)
4. 4 dps: add pathogen Fusarium culmorum
5. Monitoring germination and plant health/phytotox

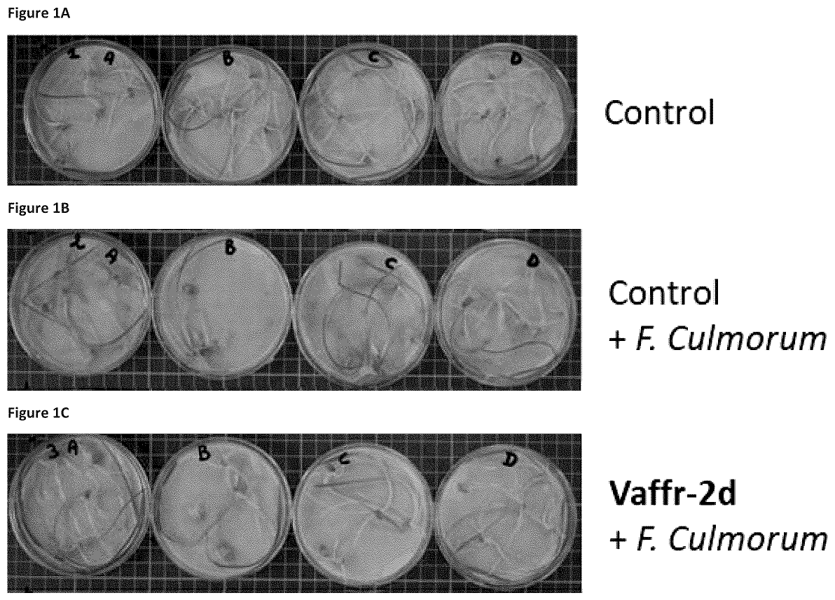

Results are shown in FIG. 1. The results show that addition of the anti-frost protein with chitinolytic activity Vaffr-2d resulted in strong inhibition of fungal growth and improved plant health (biostimulant effects compared to the control).

These results thus show that anti-frost proteins with chitinolytic activity, and in particular the anti-frost proteins described herein, can efficiently inhibit growth of pests on plants, such as fungi. Plant health can thereby be improved. Thus, anti-frost proteins can be used as plant protection agents as described herein.

### Example 2

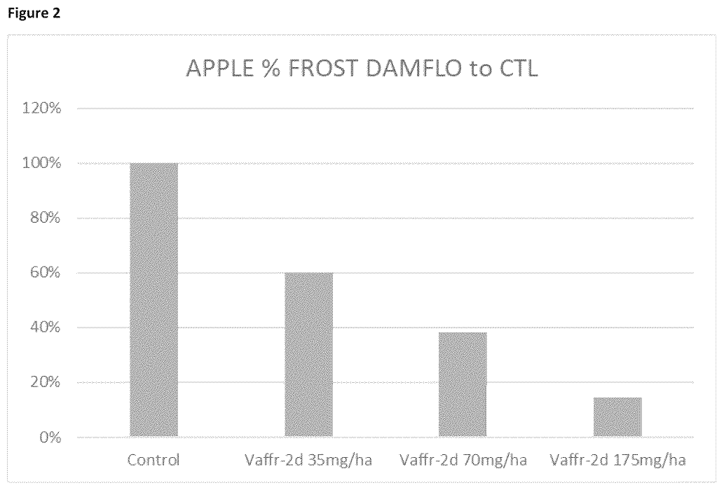

The protective activity of anti-frost proteins against frost stress was tested by foliar application of the anti-frost protein Vaffr-2d (200 l/ha of aqueous formulation comprising anti-frost protein at indicated concentrations) on apple plants and subjecting the plant to frost stress. The results are shown in the following Table 2 and FIG. 2.

TABLE 2

| | | |
|---|---|---|
| Sample | Dose | Damage |

| | | | |
|---|---|---|---|
| Control | 0 | mg/ha | 10.5% a |
| Vaffr-2d | 35 | mg/ha | 6.3% b |
| Vaffr-2d | 70 | mg/ha | 4.0% c |
| Vaffr-2d | 175 | mg/ha | 1.5% c |

a, b and c indicate increasing degrees of statistical significance at 95% level

The results show that addition of Vaffr-2d resulted in decreased frost-damage with an increase in dosage and improved plant health.

These results thus show that anti-frost proteins, and in particular the anti-frost proteins described herein, can efficiently decrease frost-damage. Plant health can thereby be improved. Thus, anti-frost proteins can be used as plant protection agents as described herein.

### Example 3

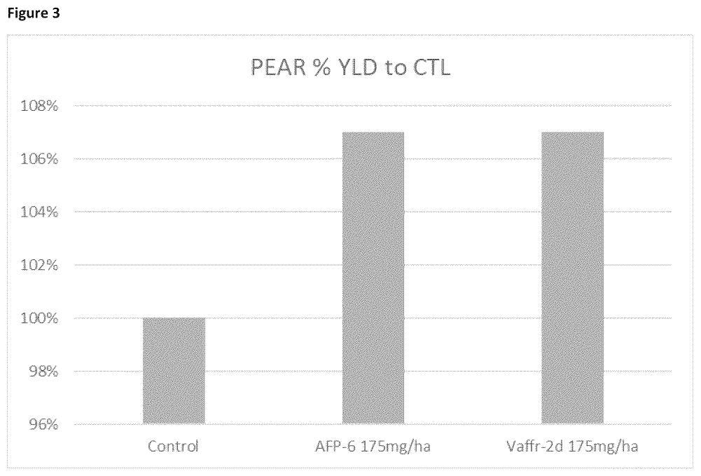

The protective activity of anti-frost proteins against frost stress was tested by foliar application of the anti-frost protein Vaffr-2d or AFP-6 on pear plants and subjecting the plants to frost stress. The results are shown in FIG. 3.

The results show that addition of Vaffr-2d or AFP-6 resulted in increased pear yield compared to control.

These results thus show that anti-frost proteins, and in particular the anti-frost proteins described herein, can efficiently decrease frost-damage. Plant health can thereby be improved. Thus, anti-frost proteins can be used as plant protection agents as described herein.

### Example 4

Different plants were subjected to different kinds of abiotic stresses with or without foliar application of Vaffr-2d or AFP-6.

Setup for salt stress experiments:
plants are drilled in trays
germination at 25° C./15° ° C. (day/night) light 16 h, dark 8 h application 10 days later
2 days later replanted in pots of 8×8 cm
3 possible regimes
   direct salt stress on moment of replanting
   salt stress 4 days after replanting
   salt stress 7 days after replanting
salt stress=100 mL of a solution of 120 g/L NaCl

Results are shown in the following Table 3:

TABLE 3
Salt stress rice

| | Plant length (cm) | % fytotox (0-100) |
|---|---|---|
| Check | 28.3 | 38.5 |
| Vaffr2d 175 mg/ha | 32.5 | 28.5 |

Drought stress rice

| | Plant length (cm) | | |
|---|---|---|---|
| | normal | drought | flooding |
| Check | 36.1 | 18.9 | 28.1 |
| Vaffr2d 175 mg/ha | 41.3 | 19.8 | 39.7 |

Drought stress corn

Plant length (cm)

| | normal | drought |
|---|---|---|
| Check | 39.1 | 21.2 |
| AFP6 70 mg/ha | 39.6 | 24.1 |

Drought stress barley

| | Plant length (cm) | | |
|---|---|---|---|
| | normal | drought | Vitality drought stress (0-100%) |
| Check | 33 | 19.7 | 55 |
| AFP6 70 mg/ha | 37.5 | 19.8 | 72 |

These results show that anti-frost proteins, and in particular the anti-frost proteins described herein, can efficiently protect plants from different abiotic stress, such as salt, drought or flooding stress. Plant health can thereby be improved. Thus, anti-frost proteins can be used as plant protection agents as described herein.

### Example 5

To investigate the protective activity of the enzyme AFP-6 against fungal infection, two greenhouse trials were performed.

For both trials, seeds were coated in a in Satec ML2000, 8 l/ton coater according to the following steps (which can be extrapolated to, e.g., commercial machines):
Required amount of seeds in the batch coater (min. 50 g and max. 2 kg in the Satec ML2000)
Start dosing—takes about 10 to 15 seconds
Then leave to dry for another 15 seconds while the machine is running
Unload the machine
Rotation speed rotor: between 500 and 1000 RPM
Rotation speed spinning disc: between 2100 and 3000 RPM
Coating temperature during the entire process: +/−20° C.
Total with loading, dosing, drying, and unloading included the coating process takes about 1 minute.

Coating was performed using 0.07 g of AFP-6 (produced by Fraunhofer-Gesellschaft) in 8 l of water comprising 8% (v/v) PVOH (i.e., in 7.36 l water+640 ml PVOH) per 1 ton of seeds. Thus, the final concentration of AFP-6 after coating was 0.07 g/ton seeds. Polyvinyl alcohol (PVOH) is a standard sticker used in commercial seed treatments. The untreated control was only water and 8% PVOH, no active ingredient was added. Four seeds per pot were sown in 6 repetitions. In the second trial, seeds were alternatively coated with Difend extra (25 g/l difeconazole and 25 g/l fludioxinil) at a concentration of 2 l/ton for comparison. Difend extra is a commercially available seed treatment.

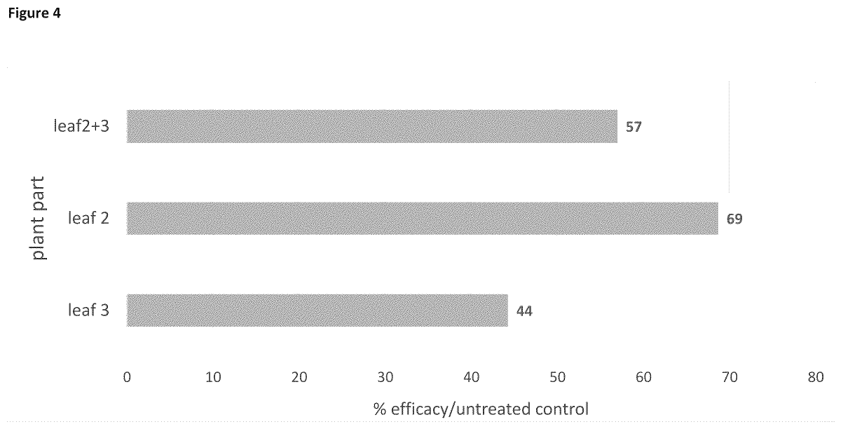

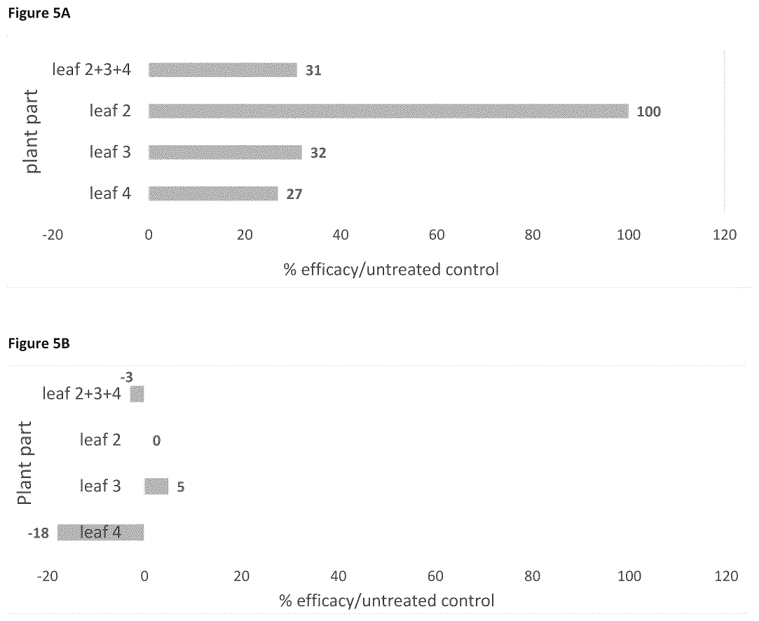

The first trial was performed with the wheat variety Benchmark. Two weeks after sowing, heavily infected wheat plants with brown rust (Puccinia triticina infection) were placed in the trial to mimic natural infections. After 8 days, the percentage of brown rust spots in the treated and untreated objects was determined on the 3 upper leaves of the plants. Results of the enzyme treated objects were compared with the untreated control and the percentage efficacy of the enzyme compared with the untreated control was calculated based on the infection percentage. The youngest leaf (leaf 1) was not infected in treated and untreated control. Thus, leaf 2 and 3 were assessed. Compared with untreated control, leaf 2 was 69% less infected and leaf 3 44% less infected (see FIG. 4). This is an average efficacy of 57% less infection for both leaves in variety Benchmark, coated with 0.07 g/ton enzyme AFP-6.

For the second trial the wheat variety Keitum was used. Two weeks after sowing, the plants were inoculated with brown rust spore suspension (concentration of 3×10⁵ spores/ml) until run off. The percentage of brown rust spots was assessed 12 days after the infection as described above on the 4 upper leaves. Results of the AFP-6- or Difend extra-treated objects were compared with the untreated control and the percentage efficacy of the treated compared with the untreated control was calculated based on the infection percentage. The youngest leaf (leaf 1) was not infected in treated and untreated control. Thus, leaf 4-3 and 2 were scored for brown rust. Comparing the AFP-6-treated plants with the untreated control, leaf 2 was 100% less infected and leaf 3 32% less infected. The oldest leaf (4) was 27% more protected. This was an average efficacy of 31% for all leaves in variety Keitum, coated with 0.07 g/ton enzyme AFP-6 (see FIG. 5A). No or only minor protection was observed for the seeds coated with Difend extra, despite the much higher concentrations used (see FIG. 5B).

These results showed that seed treatment with AFP-6 protected plants from fungal infection with Puccinia triticina, and that protection by AFP-6 was far superior to protection by the commercially available seed treatment Difend extra. Since protection was achieved by treatment of seeds with AFP-6 (i.e., by indirect fungicidal application), since the AFP-6 concentration used was very low, and since younger leaves were better protected than older leaves, these results suggested that the enzyme AFP-6 exerted protective effects by inducing plant immunity (“indirect fungicidal effect”) on both wheat varieties tested.

### Example 6

Two further trials were performed to investigate whether the enzyme AFP-6 can further improve plant protection against leave diseases when the plants are treated with AFP-6 in addition to the standard chemical treatment. These trials were conducted in practical field circumstances, wherein wheat was treated by farmers with a chemical seed treatment (Difend extra, 25 g/l difeconazole and 25 g/l fludioxinil, at a concentration of 2 l/ton) and at least 2 foliar treatments were done with chemicals in the season: T1=BBCH 32 and T2=BBCH 39. For T1 Protendo, prothioconazole 300 g/l EC (400 ml/ha) and for T2 Velogy era EC 75 g/l benzovindiflupyr+150 g/l prothioconazole (1000 ml/ha) were used.

Since the earlier experiments had suggested that enzyme AFP-6 exerted protective effects by inducing plant immunity, in both trials the enzyme AFP-6 was added as early as possible in the season, before plants were infected by fungal pathogens. The trials were drilled at 400 seeds/m². One plot was 24 m² and every object had 4 repetitions. In every plot, 4 plants were assessed according the EPPO guidelines PP(1)/026(4) for foliar and ear diseases on cereals. The percentage infection of leave diseases was scored on the 3 upper leaves of the plant. Results: abbott. The average percentage infection of the 16 plants was calculated (4 plants/plot). The percentage of efficacy of the pest severity was calculated in function of the chemical coated object with Difend extra (in case of the first trial as described below) or the object without TO treatment (in case of the second trial as described below).

In the first trial the enzyme AFP-6 was used at a concentration of 0.07 g/ton as a seed treatment. The variety in this trial was Keitum. The control was coated with the chemical seed treatment Difend extra (25 g/l difeconazole and 25 g/l fludioxinil) at a concentration of 2 l/ton. Seeds were coated in a in Satec ML2000, 8 l/ton coater as described for Example 5 above. Coating with the enzyme was performed using 0.07 g of AFP-6 (produced by Fraunhofer-Gesellschaft) in 8 l of water comprising 8% (v/v) PVOH (i.e., in 7.36 l water+640 ml PVOH) per 1 ton of seeds. Thus, the final concentration of the enzyme after coating was 0.07 g/ton seeds. Polyvinyl alcohol is a standard sticker used in commercial seed treatments. Leaf diseases were assessed 169 days after sowing and compared with the object coated with the chemical Difend extra.

In the second trial it was investigated whether an early treatment with AFP-6 before BBCH 32 (i.e., a “TO” treatment at BBCH 30) can further improve plant protection against leave diseases. The variety Ragnar was used in this trial. Seeds did have a chemical seed treatment with defend extra. Plants were sprayed with a concentration of 0.07 g/l AFP-6 in a water formulation using 1 l/ha. The foliar treatment was done in BBCH 30. Plants were assessed for leaf diseases 13 days after the TO treatment and compared with the object without TO treatment.

Both trials were assessed at the same day. Only leaf 3 showed infection with the leaf disease-causing fungus Septoria tritici. In both trials it was observed that enzyme AFP-6 improved plant protection. As a seed treatment in a concentration of 0.07 g/ton seeds in variety Keitum (first trial), the efficacy of the enzyme AFP-6 was 24% compared with the chemical seed treatment Difend extra. In the variety Ragnar (second trial), a TO treatment in BBCH 30 showed an efficacy of 25% compared with no TO treatment. These results, obtained with low concentrations of AFP-6, confirm that AFP-6 exerts protective effects by inducing plant immunity.

### Example 7

Further trials were performed to confirm that AFP-6 and Vaffr-2d exert protective effects by inducing plant immunity. In this regard it is important to note that different plant immunity pathways may be triggered depending on the kind of stress the plant is exposed to:

Plant hormones jasmonic acid and salicylic acid play an important role as regulators of plant defense against biotic and abiotic stress. An effect on plant immunity against biotrophic pathogens is dependent on salicylic acid. For necrotrophic pathogens, jasmonic acid is more important (Glazebrook, 2005: “Contrasting mechanisms of defense against biotrophic and necrotrophic pathogens”; Annual review of phytopathology, 43, 205; doi: doi. 10.1146/annurev.phyto.43.040204.135923). Also different types of abiotic stress will trigger different pathways in the plant (Peleg, 2011: “Hormone balance and abiotic stress tolerance in crop plants”; Current opinion in plant biology, 14(3), 290-295; doi: https://doi.org/10.1016/j.pbi.2011.02.001).

Therefore, for the following trials, 4 different genes were selected to prove AFP-6 and Vaffr-2d are having effects on plant immunity, i.e. the LOX, OPR3, PR1-3 and PR1-17 genes. The LOX and OPR3 genes are upregulated in order to activate the jasmonic acid pathway (Chini, 2018: “An OPR3-independent pathway uses 4,5-didehydrojasmonate for jasmonate synthesis”; Nature chemical biology, 14(2), 171-178; doi: https://doi.org/10.1038/nchembio.2540) (León, 1999: “Molecular biology of jasmonic acid biosynthesis in plants”; Plant physiology and Biochemistry, 37(5), 373-380; doi: https://doi.org/10.1016/S0981-9428(99)80043-6). As indicated above, jasmonic acid is an important plant hormone that regulates plant immunity against necrotrophic pathogens. PR1-3 and PR1-17 are PR proteins produced by plants to defend themselves against biotrophic pathogens. The production of these compounds is linked with the plant hormone salicylic acid (Van Loon, 1999: “The families of pathogenesis-related proteins, their activities, and comparative analysis of PR-1 type proteins”; Physiological and molecular plant pathology, 55(2), 85-97; doi: https://doi.org/10.1006/pmpp. 1999.0213).

### AFP-6

Lab trials were performed to investigate the plant immunity inducing potential of AFP-6. Seeds were coated in a in Satec ML2000, 8 l/ton coater with 2 concentrations of AFP-6 on wheat (variety chamsin) according to the steps outlined in Example 5 above. Coating was performed using 0.1 g or 0.175 g of AFP-6 (produced by Fraunhofer-Gesellschaft) in 8 l of water comprising 8% (v/v) PVOH (i.e., in 7.36 l water+640 ml PVOH) per 1 ton of seeds. Thus, the final concentration of the enzyme after coating was 0.1 g/ton and 0.175 g/ton seeds. PVOH is a standard sticker used in commercial seed treatments. The untreated control was coated with water and 8% PVOH. Five seeds were placed on moister filter paper in petri dishes. The seeds were germinated in an incubator on 20° C. in a 12 h light/12 h dark regime. For each object, 3 repetitions were performed and this for each sampling timepoint. Samples of the seedlings were taken at different timepoints after ‘sowing’ on filter paper: 4-7-9 and 14 days.

Total mRNA was isolated from plant tissue using of TRI reagent (Sigma-Aldrich, St. Louis, MO, USA). Total RNA concentration was quantified using the NanoDrop method (ND-1000 spectrophotometer, Thermo Scientific, USA). cDNA was generated from 1 mg of RNA of each sample by using an anchored oligo(dT)18 primer and a hexamer primer according to the instructions of the Transcriptor first-strand cDNA synthesis kit (Roche). The qRT-PCRs were performed on a StepOnePlus™ PCR machine (Applied Biosystems, UK) using qPCR Mastermix Plus for SYBR Green I (Eurogentec, San Diego, CA, USA) with primers targeting the genes of interest. Expression levels of transcripts from the various samples were normalized to the standardized reference genes actin and ubiquitin and PCR data were analyzed using the 2-ΔCt method.

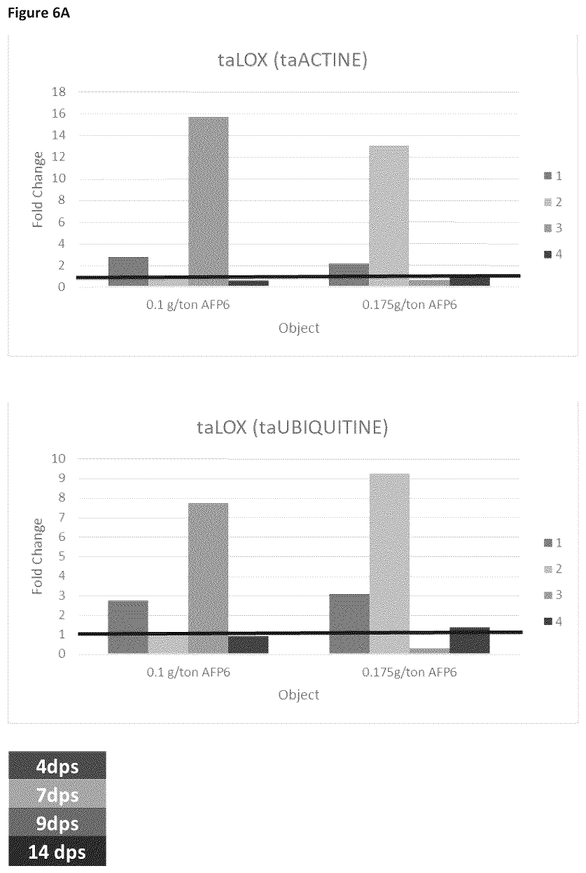

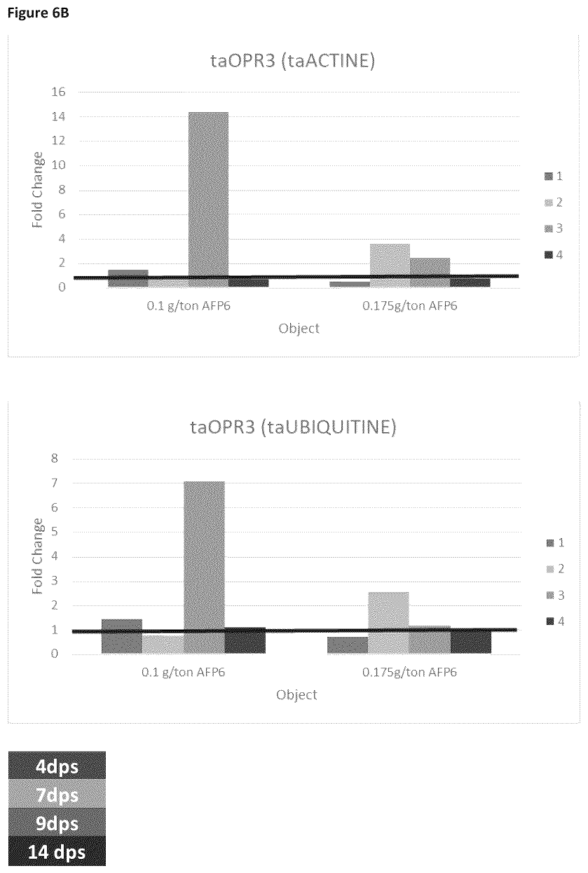

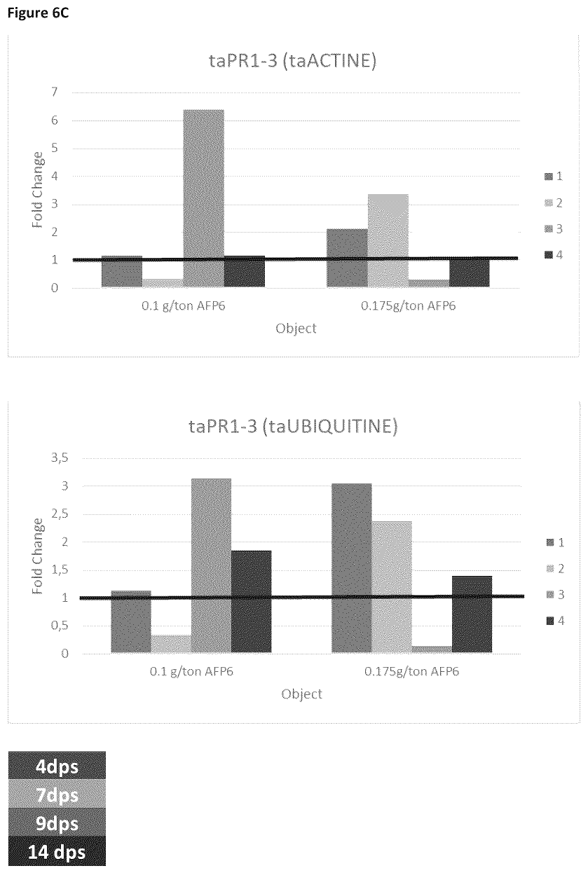

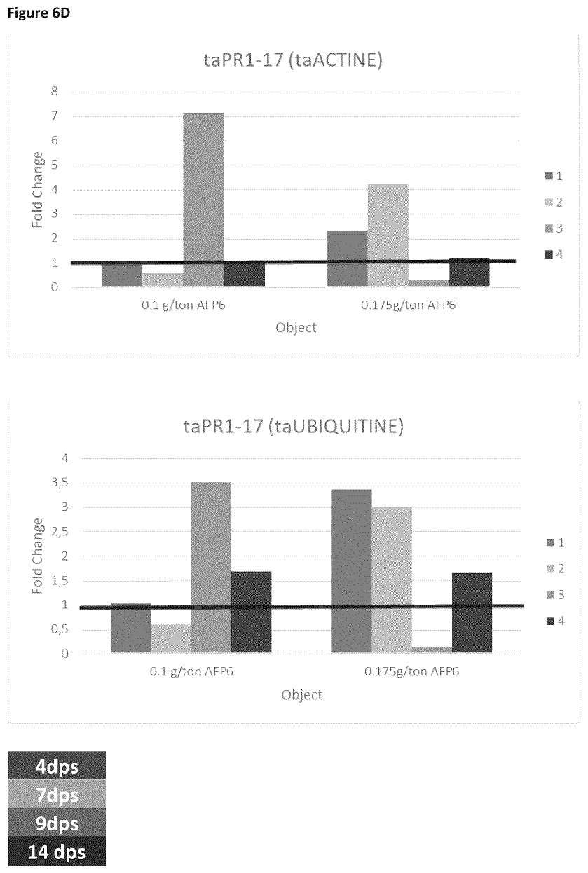

The results are shown in FIGS. 6A-6D. These results show that AFP-6 upregulates both the jasmonic acid pathway (LOX & OPR3; see FIG. 6A and FIG. 6B, respectively) and the salicylic acid pathway (PR1-3 & PR1-17; see FIG. 6C and FIG. 6D, respectively). This means that AFP-6 primes plant immunity both against necrotrophic and biotrophic pathogens (i.e., against different kinds of pests) and different kinds of abiotic stress.

### Vaffr-2d

A greenhouse trial was performed to investigate the plant immunity inducing potential of Vaffr-2d. Seeds were coated in a in Satec ML2000, 8 l/ton coater with 1 concentration of Vaffr-2d on wheat (variety chamsin) according to the steps outlined in Example 5 above. Coating was performed using 0.175 g of Vaffr-2d in 8 l of water comprising 8% (v/v) PVOH (i.e., in 7.36 l water+640 ml PVOH) per 1 ton of seeds. Thus, the final concentration of the enzyme after coating was 0.175 g/ton seeds. PVOH is a standard sticker used in commercial seed treatments. The untreated control was coated with water and 8% PVOH. Forty seeds/pot were sown in potting soil and 4 pots were used. Samples of the plants were taken at different timepoints after sowing: 6-7-8 and 11 days. The extraction of mRNA and the qRT-PCRs were performed as described for AFP-6 above. The same genes were selected as in the AFP-6 trial.

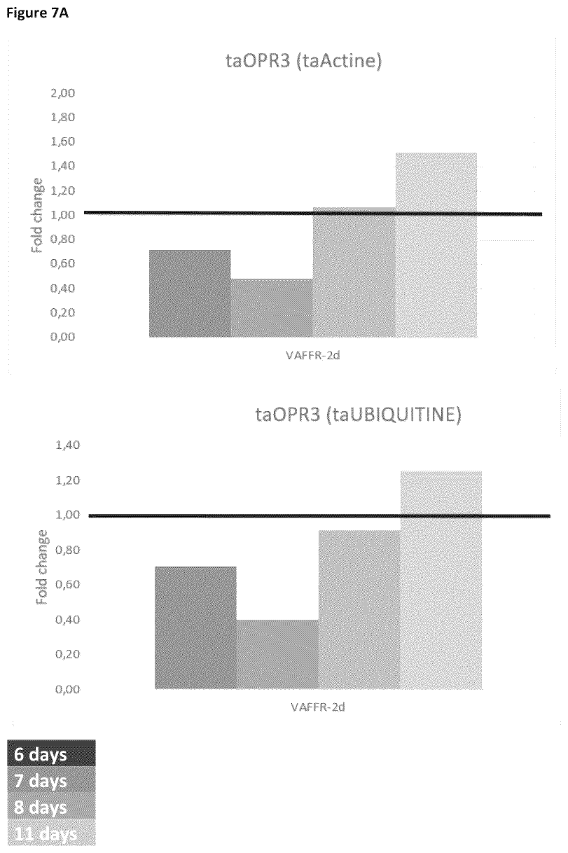

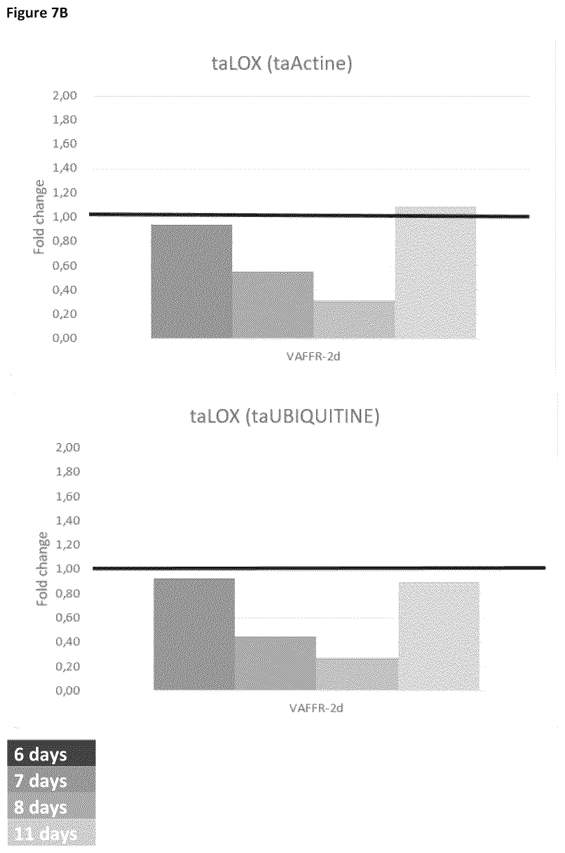

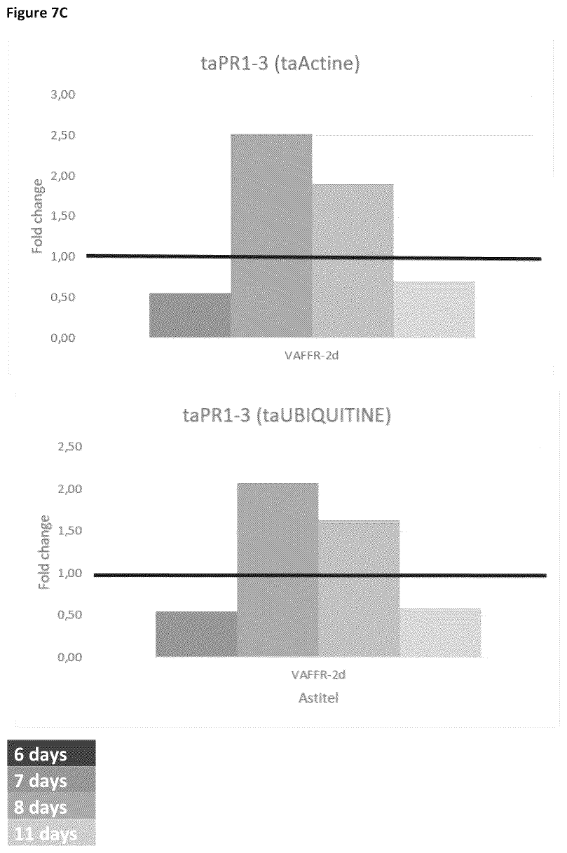

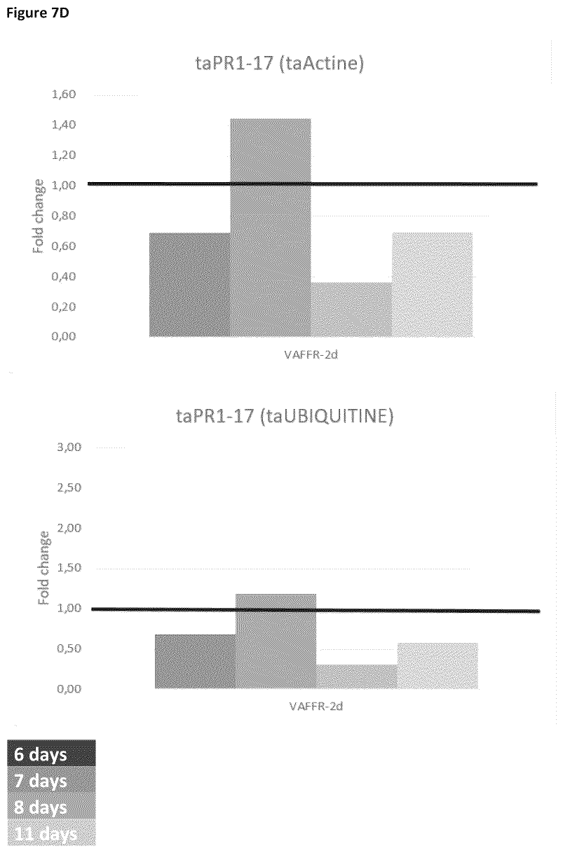

The results are shown in FIGS. 7A-7D. These results show that also Vaffr-2d shows upregulation of both the jasmonic acid pathway and the salicylic acid pathway. These results show that by using Vaffr-2d, e.g. as a preventive treatment, plant immunity can be primed against various biotic and abiotic stresses.

### INDUSTRIAL APPLICABILITY

The anti-frost proteins described herein, and in particular for a use thereof in plant protection, as well as the related products and uses described herein can be applied, for example, to commercial plant protection agents e.g. for use in agriculture. The present disclosure is thus industrially applicable.

1. A composition comprising at least one anti-frost protein, the anti-frost protein comprising a first amino acid sequence that is at least 70%, such as 100%, identical to an amino acid sequence selected from the group of SEQ ID NOs: 1-2.

2. The composition according to claim 1, wherein the anti-frost protein comprises the amino acid sequence according to SEQ ID NO: 1.

3. The composition according to claim 1 or 2, wherein the anti-frost protein comprises the amino acid sequence according to SEQ ID NO: 2.

4. The composition according to any one of claims 1-3, wherein the anti-frost protein essentially consists of a first amino acid sequence that is at least 70%, such as 100%, identical to an amino acid sequence selected from the group of SEQ ID NOs: 1-2.

5. The composition according to any one of claims 1-4, wherein the anti-frost protein consists of a first amino acid sequence that is at least 70%, such as 100%, identical to an amino acid sequence selected from the group of SEQ ID NOs: 1-2.

6. The composition according to any one of claims 1-5 that is a plant protection agent.

7. Use of a composition comprising at least one anti-frost protein as a plant protection agent.

8. Use of the composition according claim 7, wherein the composition is according to any one of claims 1-6.

9. Use of the composition according to claim 7 or 8 as a plant protection agent against abiotic stress and/or a pest.

10. Use of the composition according to any one of claims 7-9, wherein the plant protection agent is against abiotic stress, wherein the abiotic stress optionally is frost stress or drought stress.

11. Use of the composition according to any one of claims 7-10, wherein the plant protection agent is against abiotic stress, wherein the abiotic stress is frost stress.

12. Use of the composition according to any one of claims 7-11, wherein the plant protection agent is against abiotic stress, wherein the abiotic stress is drought stress.

13. Use of the composition according to any one of claims 7-9, wherein the plant protection agent is against a pest, wherein the pest optionally is an organism that contains chitins, such as a fungus or an insect.

14. Use of the composition according to any one of claim 7-9 or 13, wherein the plant protection agent is against a pest, wherein the pest is a fungus.

15. Use of a composition according to any one of claims 13-14, wherein the fungus is a Fusarium or Septoria species.

16. Use according to any one of claims 7-15, wherein the plant is an arable crop, fruit-bearing plant or vegetable.

17. A method of protecting a plant from abiotic stress and/or a pest, the method comprising the application of a composition comprising at least one anti-frost protein on the plant or a part thereof.

18. The method of protecting a plant according to claim 17, wherein the composition is according to any one of claims 1-6.

19. The method of protecting a plant according to any one of claim 17 or 18, wherein the plant or plant part is soaked in the composition.

20. The method of protecting a plant according to any one of claims 17-19, wherein the composition is applied by spraying to a surface of the plant or part of the plant, such as a seed, for example a coated a seed.

21. The method according to any one of claims 17-20, wherein the method is for protecting the plant from abiotic stress, wherein the abiotic stress optionally is frost stress or drought stress.

22. The method according to any one of claims 17-21, wherein the method is for protecting the plant from abiotic stress, wherein the abiotic stress optionally is frost stress.

23. The method according to any one of claims 17-22, wherein the method is for protecting the plant from abiotic stress, wherein the abiotic stress optionally is drought stress.

24. The method according to any one of claims 17-20, wherein the pest is an organism that contains chitin, such as a fungus or an insect.

25. The method according to any one of claim 17-20 or 24, wherein the pest is a fungus.

26. The method according to claim 24-25, wherein the fungus is a Fusarium or Septoria species.

27. The method according to any one of claims 17-26, wherein the plant is an arable crop, fruit-bearing plant or vegetable.
